(19) United States
(12) Patent Application Publication
(10) Pub. No.: US 2010/0172935 A1
(43) Pub. Date: Jul. 8, 2010

(54) Method for viral inoculation of the vestibular region of the nares

(76) Inventor: John A. Willimann, Chevy Chase, MD (US)

Correspondence Address: ROBERT M. DOWNEY, P.A., 6751 N. FEDERAL HWY., SUITE 300, BOCA RATON, FL, 33487, US

(21) Appl. No.: 12/658,093

(22) Filed: Feb. 1, 2010

Related U.S. Application Data
(63) Continuation-in-part of application No. 11/604,015, filed on Nov. 22, 2006.
(60) Provisional application No. 60/739,989, filed on Nov. 23, 2005.

(51) Int. Cl. A61K 39/12; A61P 31/12; A61P 37/04

(52) U.S. Cl. 424/204.1

(57) ABSTRACT

A method of inoculation of the vestibular region of the nares with a virus, provides the steps of: applying the virus exclusively to the anterior vestibular region of the nares; and avoiding penetration of the virus beyond the vestibular region during application.

This application is a Continuation-In-Part application of co-pending patent application Ser. No. 11/604,015 filed on Nov. 22, 2006 which was based on provisional patent application Ser. No. 60/739,989 filed on Nov. 22, 2005.

### BACKGROUND OF THE INVENTION

1. Field of the Invention

The invention relates to a method of inoculating the vestibular region of the nares with virus to ascertain whether acquisition and/or prevention of transmission of infection may occur.

2. Discussion of the Related Art

The nose is believed to be the primary incubator of various pathogenic organisms including bacteria, virus, and mold. However, it is the accepted belief in the medical community that inoculation of virus exclusively to the vestibular region of the nose will not play a major role in whether or not, and the degree to which acquisition of infection may occur.

Because viruses are acellular and do not use ATP (Adenosine Triposphate), they must utilize the machinery and metabolism of a host cell to reproduce. For this very reason, viruses are called obligate intracellular parasites. Before a virus has entered a host cell, it is called a virion (i.e., a package of viral genetic material). Virions (i.e., infectious viral particles) can be passed from host to host either through direct contact or through a vector, or carrier. After entering the cell, the virus's genetic material begins the destructive process of taking over the cell and forcing it to produce new viruses.

When the virus has taken over the cell, it immediately directs the host to begin manufacturing the proteins necessary for virus reproduction. The host produces three kinds of proteins: early proteins, which are enzymes used in nucleic acid replication; late proteins, which are proteins used to construct the virus coat; and lytic proteins, which are enzymes used to break open the cell for viral exit. The final viral product is assembled spontaneously, that is, the parts are made separately by the host and are joined together by chance. This self-assembly is often aided by molecular chaperones, or proteins made by the host that help the capsid parts come together.

The new viruses then leave the cell either by exocytosis or by lysis. Envelope-bound animal viruses instruct the host's endoplasmic reticulum to make certain proteins, called glycoproteins, which then collect in clumps along the cell membrane. The virus is then discharged from the cell at these exit sites, referred to as exocytosis. On the other hand, bacteriophages must break open, or lyse, the cell to exit. To do this, the phages have a gene that codes for an enzyme called lysozyme. This enzyme breaks down the cell wall, causing the cell to swell and burst. The new viruses are released into the environment, killing the host cell in the process. Viruses, very different from bacteria, require targeted host cells to effectively replicate.

The interior of the nasal passages, starting at the entrance into the nose at the nares, can be divided into distinct regions based on the type of nasal epithelial cells that line those regions. The nasal vestibule resides at the foremost position, just inside the nares. Keratinized stratified squamous epithelium lines the anterior two-thirds of the vestibule. In the underlying dermis, sebaceous and sweat glands, and hair follicles are present. These cells do not secrete, contain cilia nor express on their surfaces the cell-surface receptor, ICAM-1, to which all of the major group human rhinoviruses (including types 9 and 39) exclusively bind in order to initiate infection. This is the region focused on by the present invention. The posterior third of the nasal vestibule is lined by non-keratinized stratified squamous epithelium, which is similar to keratinized squamous epithelium except that the upper cell layers do not keratinize. At its posterior annular ring, which is lined by a small band of transitional cells, the vestibule opens into the main body of the nasal cavity, including the nasal turbinates, paranasal sinuses, and parts of the anterior, posterior, and lateral walls of the nasopharanx. This region, which constitutes the vast majority of the nasal interior, is lined by the nasal mucosa comprised of pseudostratified columnar ciliated epithelium with goblet cells scattered among ciliated cells. This region is responsible for the majority of secretions in the nose and its cells abundantly express the rhinovirus binding molecule, ICAM-1, on their surfaces:

Methods of inoculation with virus for the purpose of initiating infection employed by researchers skilled in the art is consistent with the accepted belief that viral infection requires deposition of virus inoculum on the mucosal region of the nasal cavities that are lined by pseudostratified ciliated and secretory columnar epithelial cells. This region represents the focus of viral spread and the symptoms of the common cold that include excess mucus production, swelling and inflammation. Access to these regions of the inner nasal surfaces is typically gained by the use of nasal dropper or pipette administration which maximizes the ability of the liquid inoculum to distribute itself in these areas. Nasal drop administration with the head held back largely bypasses the anterior portion of the nasal vestibule, with the exception of some run-out that may occur when the head regains its normal position.

Embedded within the currently held beliefs of the larger scientific community, and those intimately involved in respiratory epithelial cell research, lies the presumption that upper respiratory virus infection requires inoculation of the interior nasal mucosal epithelium. Consistent with the beliefs of the medical and research communities, and absent of the findings outlined in this disclosure of my invention, I too would have joined the greater scientific community in rejecting the notion that exclusive inoculation of the anterior nasal vestibule would be adequate to initiate viral infection.

Although the methods employed by others may have passively included inoculation of the vestibular region, those methods were and are consistent with the currently-accepted techniques of delivering inocula to the inner nasal cavity in order to achieve upper respiratory infection in test subjects. (see Al-Nakib et al (1989) Antimicrobial Agents and Chemotherapy, April 1989, Vol. 33, No. 4, p. 552-525, and Farr et al (1987) Antimicrobial Agents and Chemotherapy, August 1987, Vol. 31, No. 8, p. 1183-1187) The prior art teachings are in direct contradiction to the unique invention herein which involves selective viral inoculation of the anterior vestibule to the exclusion of all other nasal regions. Furthermore, given what is known about the nature of the cells lining the anterior vestibule, it appears to be neither obvious nor predictable based on current common practice or belief that exclusive viral inoculation of the anterior vestibule would result in infection.

Recent efforts to prevent infection have revolved around attempts to interrupt direct contact transmission by inactivating the pathogen before it reaches the pseudocolumnar epithelium. These attempts have included inactivation of the virus on the hands, or prevention of attachment of virus to the respiratory epithelium. The normal ciliary clearance of foreign material from the nose poses a formidable barrier to the use of the intranasal strategies. Inactivation of bacteria, virus, and mold in the vestibule, where ciliary clearance is not an issue, provides a strategy to overcome this barrier.

The potential utility of this strategy requires an assessment of whether a virus inoculated onto the vestibular epithelium actually contributes to the transmission of viral infection.

### DETAILED DESCRIPTION OF THE PREFERRED EMBODIMENT

Healthy volunteers who were found to have serum neutralizing antibody titers of <1:4 to rhinovirus type 39 were enrolled to validate the viability of the method described herein.

The description of the method of viral inoculation of the vestibular region of the nares is set forth below.

The challenge virus used in this study was a safety tested pool of rhinovirus type 39. This pool has a starting titer of approximately 103.8 TCID50/mL.

Virus challenge: On the day of the virus challenge, each volunteer had a symptom score evaluated in an interactive interview with the study coordinator to assure that all were asymptomatic and had a blood specimen collected for serologic testing. Each volunteer then had approximately 160 TCID50 of RV39, contained in a volume of 10 μl, placed into the “cup” formed by the thumb and first two fingers of the right hand. The volunteers were instructed to spread the virus over the fingertips with the thumb of the right hand. When the virus challenge had dried (˜10 min), each volunteer intentionally inoculated the anterior nares with the first and second fingers of the right hand. This procedure was carefully monitored to ensure that the finger was inserted only approximately 1 cm into the nose to limit inoculation to the vestibule region of the nares.

Subjects returned to the study site daily for 5 days after the virus challenge for collection of a nasal lavage specimen for viral culture. Nasal lavage was mixed 1:4 with 4× (four times) concentrated viral collecting broth and then stored frozen until cultured. Each specimen was cultured in two tube cultures of human embryonic lung fibroblast cells (one tube of MRC-5 and one tube of WI-38). These cultures were incubated on roller drums at 33C and observed for 10 days for development of viral cytopathic effect typical of rhinovirus. Rhinovirus isolates from subjects who did not have a serum neutralizing antibody response were neutralized with antibody to RV39 to confirm that the infection was due to the challenge serotype. Serum collected prior to challenge and again approximately 18 days later was assayed for antibody to RV39 by a microtiter neutralization assay. Volunteers with rhinovirus detected in any post-challenge culture or with at least a four-fold rise in serum neutralizing antibody titer between the acute and convalescent specimens were considered infected.

Fifty percent (50%) of the volunteers challenged with RV39 in this study became infected with the challenge virus (95% CI: 0.24-0.76). Three volunteers had both virus isolation seroconversion, two volunteers had infection documented by virus isolation alone.

Conclusions: Inoculation of the vestibule of the nares resulted in infection of 50% of challenged subjects in this study. These results document the feasibility of this route of infection and suggest that inactivation of virus by virucidal treatment of the nasal vestibule will potentially have an impact on rhinovirus infections transmitted by direct contact.

What is claimed is:

1. A method for viral inoculation of humans, comprising the steps of:
applying a virus exclusively to the anterior vestibular region of the nares of a person; and
avoiding penetration of the virus beyond the vestibular region during said step of applying.

2. The method as recited in claim 1 further comprising the steps of:
collecting a daily nasal lavage specimen from the person for viral culture for at least three consecutive days;
observing the collected nasal lavage specimens for a plurality of days to determine development of viral cytopathic effect and viral infection of the person.

3. The method as recited in claim 1 wherein said step of applying the virus exclusively to the anterior vestibular region of the nares of a person further includes the steps of:
applying the virus to an applicator; and
inserting the applicator one centimeter into the nose and wiping the applicator against the vestibular region of the nares.

4. A method for viral inoculation of humans, comprising the steps of:
applying a virus exclusively to the anterior vestibular region of the nares of a person using an applicator;
avoiding penetration of the applicator and virus beyond the vestibular region during said step of applying.

5. The method as recited in claim 4 wherein said step of applying the virus exclusively to the anterior vestibular region of the nares of a person using an applicator further includes the steps of:
first applying the virus to the applicator; and
inserting the applicator one centimeter into the nose and wiping the applicator against the vestibular region of the nares to transfer the virus from the applicator to the vestibular region.
